(19) United States
(12) Patent Application Publication
(10) Pub. No.: US 2006/0088831 A1
(43) Pub. Date: Apr. 27, 2006

(54) Methods for identifying large subsets of differentially expressed genes based on multivariate microarray data analysis

(75) Inventors: Aniko Szabo, LAYTON, UT (US); Kenneth Boucher, Salt Lake City, UT (US); David Jones, Salt Lake City, UT (US); Lev Klebanov, Czech Republic (RU); Alexander Tsodikov, Sacramento, CA (US); Andrei Yakovlev, Pittsford, NY (US)

Correspondence Address: HELLER EHRMAN WHITE & MCAULIFFE LLP, 1717 RHODE ISLAND AVE, NW, WASHINGTON, DC, 20036-3001, US

(73) Assignee: UNIVERSITY OF UTAH RESEARCH FOUNDATION, SALT LAKE CITY, UT (US)

(21) Appl. No.: 10/506,767

(22) PCT Filed: Mar. 7, 2003
(86) PCT No.: PCT/US03/07103

§ 371 Date: Sep. 14, 2005

Related U.S. Application Data
(60) Provisional application No. 60/362,087, filed on Mar. 7, 2002.

(51) Int. Cl. C12Q 1/68; G06F 19/00

(52) U.S. Cl. 435/6; 702/20

(57) ABSTRACT

The present invention provides multivariate methods for identifying differentially expressed genes based on microarray expression data. An improved random search procedure involving certain probability distances is provided. The methods of this invention implement a successive elimination procedure to remove smaller subsets resulted from each step of the random search thereby establishing a larger set of differentially expressed genes.

### BACKGROUND OF THE INVENTION

1. Field of the Invention

The present invention relates in general to statistical analysis of microarray data generated from nucleotide arrays. Specifically, the present invention relates to identification of differentially expressed genes by multivariate microarray data analysis. More specifically, the present invention provides an improved multivariate random search method for identifying large sets of genes that are differentially expressed under a given biological state or at a given biological locale of interest according to the values of a probability distance calculated for numerous subsets of genes. The method of the invention provides a successive elimination procedure to remove smaller subsets resulted from each step of the random search thereby establishing a larger set of differentially expressed genes.

2. Description of the Related Art

Gene expression analyses based on microarray data promises to open new avenues for researchers to unravel the functions and interactions of genes in various biological pathways and, ultimately, to uncover the mechanisms of life in diversified species. A significant objective in such expression analyses is to identify genes that are differentially expressed in different cells, tissues, organs of interest or at different biological states. So identified, a set of differentially expressed genes associated with a certain biological state, e.g., tumor or certain pathology, may point to the cause of such tumor or pathology, and thereby shed light on the search of potential cures.

In practice, however, gene expression studies are hampered by many difficulties. For example, poor reproducibility in microarray readings can obscure actual differences between normal and pathological cells or create false positives and false negatives. The tension between the extremely large number of genes present (hence high dimensionality of the feature space) and the relatively small number of measurements also poses serious challenges to researchers in making accurate diagnostic inferences.

Existing methods for selecting differentially expressed genes are typically univariate, not taking into account the information on interactions among genes. As appreciated by an ordinary skilled molecular biologist, genes do not operate in isolation—activation of one gene may trigger changes in the expression levels of other genes. That is, genes may be involved in one or more pathways or networks. Therefore, determination of differentially expressed genes calls for consideration of covariance structure of the microarray data, in addition to, for example, mean expression levels. In this regard, however, application of well-established statistical techniques for multidimensional variable selection encounters much difficulty. This is so because, in one aspect, the small number of independent samples and the presence of outliers make the estimates on selected variables unstable for large dimensions. In other words, only small sets of genes can be meaningfully considered while a relatively large number of genes are potentially differentially expressed. It is generally impossible to compare all gene subsets and find the optimal one because the number of possible gene combinations is prohibitively large. On the other hand, if a global optimum could be found, it might be overly specific to a training sample due to overfitting. Thus, it remains a significant challenge to scale methods for identifying differentially expressed genes to deal with microarray data of high dimensional space.

Therefore, there is a need to address the difficulties in applying multivariate analysis to microarray data—a need to provide methods for identifying differentially expressed genes based on gene expression data with high dimensional feature space.

### SUMMARY OF THE INVENTION

It is therefore an object of this invention to provide multivariate methods for analyzing microarray gene expression data of high dimensional space thereby identifying differentially expressed genes. Particularly, it is an object of this invention to provide methods for identifying larger sets of differentially expressed genes by successive eliminating smaller subsets of genes identified from each step of the random search procedure.

In accordance with the present invention, there is provided methods for identifying a set of genes from a multiplicity of genes whose expression levels at a first and a second state, in a first and a second tissue, or in a first and a second types of cells are measured in replicates using one or more nucleotide arrays, thereby generating a first plurality of independent measurements of the expression levels for the first state, tissue, or type of cells and a second plurality of independent measurements of the expression levels for the second state, tissue, or type of cells. The methods comprise: (a) identifying a quality function capable of evaluating the distinctiveness between the first plurality and the second plurality; (b) forming a first predetermined number of permutations from the first and the second pluralities, dividing the permutations into a first permutated plurality and a second permutated plurality, corresponding in size, to the first and second plurality, respectively, and identifying groups of genes the size of which is a second predetermined number, wherein the values of the quality function for the group of genes in the first permutated and second permutated pluralities attain the maximum; (c) determining, from the first and second permutated pluralities, the top ad percentile of the null distribution based on a quantitative characteristic of the groups of genes; (d) identifying, based on the first and second pluralities, a subset of genes the size of which is the second predetermined number, wherein the values of the quality function for the subset of genes in the first and second pluralities attain the maximum; (e) adding to the set of genes, the subset, if the value of the quantitative characteristic associated with the subset exceeds the top α^{th} percentile of the null distribution; and (f) removing from the first and second pluralities, all measurements on the subset, if the maximum value of the quality function associated with the subset exceeds the top α^{th} percentile of the null distribution, and repeating steps (d)-(f) until no more measurements are left in the first and second pluralities or the value of the quantitative characteristic associated with the subset does not exceed the top α^{th} percentile of the null distribution.

According to the present invention, in certain embodiments, the states is may be biological states, physiological states, pathological states, and prognostic states. In other embodiments, the tissues may be normal lung tissues, cancer lung tissues, normal heart tissues, pathological heart tissues, normal and abnormal colon tissues, normal and abnormal renal tissues, normal and abnormal prostate tissues, and normal and abnormal breast tissues. In yet other embodiments, the types of cells may be normal lung cells, cancer lung cells, normal heart cells, pathological heart cells, normal and abnormal colon cells, normal and abnormal renal cells, normal and abnormal prostate cells, and normal and abnormal breast cells. In still other embodiments, the types of cells may be cultured cells and cells isolated from an organism.

In one embodiment of this invention, the quality function is represented by a probability distance between random vectors. In another embodiment, the probability distance function is selected from the group consisting of the Mahalanobis distance and the Bhattacharya distance. In yet another embodiment, the probability distance function is defined as:
N(μ,ν)=2∫_{R}_{^{d}}∫_{R}_{^{d}}L(x,y)dμ(x)dν(y)−∫_{R}_{^{d}}∫_{R}_{^{d}}L(x,y)dμ(x)dμ(y)−∫_{R}_{^{d}}∫_{R}_{^{d}}L(x,y)dν(x)dν(y)
where μ and ν are two probability measures defined on the Euclidean space, and L(x, y) is a strictly negative definite kernel. In still another embodiment, the negative definite kernel is combined with the Euclidean distance between x and y to form a composite kernel function.

According to one embodiment, the quantitative characteristic is selected from the group consisting of an associated probability distance, a test set classification rate, and a cross-validation classification rate.

According to another embodiment, the formation of the permutations further comprises: (i) shifting the measurements in the first and second pluralities such that the marginal means thereof share the same true mean; and (ii) randomly permuting the resulting shifted measurements thereby forming a null-distribution of permutations.

According to yet another embodiment, the identifying further comprises: (i) calculating the values of the quality function for the subset of genes in the first and second pluralities thereby evaluating the distinctiveness of the first and second pluralities; and (ii) substituting a gene in the subset with one outside of the subset, thereby generating a new subset, and repeating step (i), keeping the new subset if the distinctiveness increases and the original subset if otherwise; and (iii) repeating steps (i) and (ii) for a fourth predetermined number of times.

According to still another embodiment, the identifying further comprises: (i) randomly dividing the first and the second pluralities into v groups of an approximate equal size; (ii) removing one of the v groups from the first and second pluralities and identifying, from the resulting reduced first and second pluralities, a subset of genes for which the value of the quality function attains the maximum; and (iii) repeating step (ii) for each of the v groups thereby obtaining v subsets of genes.

In various embodiments of the invention, the nucleotide arrays may be arrays having spotted thereon cDNA sequences and/or arrays having synthesized thereon oligonucleotides.

### BRIEF DESCRIPTION OF DRAWINGS

FIG. 1 shows the properties of the optimal subsets of genes identified in a computer simulation study using a random search method with a successive elimination procedure according to one embodiment of the invention.

FIG. 2 shows the properties of the optimal subsets of genes identified in an expression analysis of colon cancer cells using a random search procedure with a successive elimination procedure according to one embodiment of the invention.

FIG. 3 shows the estimates of the null-distributions based on the associated probability distance (the top panel), the test set classification rate (the bottom panel, the curve on the left), and the cross validation classification rate (the bottom panel, the curve on the right) for the 5-element optimal subset of genes in a “no-difference” dataset generated by a resampling procedure according to one embodiment of the invention.

### DETAIL DESCRIPTIONS OF DISCLOSURE

Definition

As used herein, the term “microarray” refers to nucleotide arrays; “array,” “slide,” and “chip” are used interchangeably in this disclosure. Various kinds of nucleotide arrays are made in research and manufacturing facilities worldwide, some of which are available commercially. There are, for example, two kinds of arrays depending on the ways in which the nucleic acid materials are spotted onto the array substrate: oligonucleotide arrays and cDNA arrays. One of the most widely used oligonucleotide arrays is GeneChip™ made by Affymetrix, Inc. The oligonucleotide probes that are 20- or 25-base ong are synthesized in silico on the array substrate. These arrays tend to achieve high densities (e.g., more than 40,000 genes per cm²). The cDNA arrays, on the other hand, tend to have lower densities, but the cDNA probes are typically much longer than 20- or 25-mers. A representative of cDNA arrays is LifeArray made by Incyte Genomics. Pre-synthesized and amplified cDNA sequences are attached to the substrate of these kinds of arrays.

Microarray data, as used herein, encompasses any data generated using various nucleotide arrays, including but not limited to those described above. Typically, microarray data includes collections of gene expression levels measured using nucleotide arrays on biological samples of different biological states and origins. The methods of the present invention may be employed to analyze any microarray data; irrespective of the particular microarray platform from which the data are generated.

Gene expression, as used herein, refers to the transcription of DNA sequences, which encode certain proteins or regulatory functions, into RNA molecules. The expression level of a given gene refers to the amount of RNA transcribed therefrom measured on a relevant or absolute quantitative scale. The measurement can be, for example, an optic density value of a fluorescent or radioactive signal, on a blot or a microarray image. Differential expression, as used herein, means that the expression levels of certain genes are different in different states, tissues, or type of cells, according to a predetermined standard. Such standard maybe determined based on the context of the expression experiments, the biological properties of the genes under study, and/or certain statistical significance criteria.

The terms “vector,” “probability distance,” “distance,” “the Mahalanobis distance,” “the Euclidean distance,” “feature,” “feature space,” “dimension,” “space,” “type I error,” “type II error,” “ROC curve,” “permutation,” “random permutation,” and “null distribution” are to be understood consistently with their typical meanings established in the relevant art, i.e. the art of mathematics, statistics, and any area related thereto. For example, a set of microarray data on p distinct genes represents a random vector X=X₁, . . . , Xₚ with mutually dependent components.

Random Search to Identify Subsets of Genes of a Predetermined Size

Suppose two tissues, types of cells, or biological states are of interest, one of which corresponds to the normal physiology while the other implicates certain pathology such as tumor. The distinctiveness of these two tissues, types of cells, or states can be evaluated by microarray experiments in which the expression levels of all the genes (up to thousands measured on a single chip or slide as made possible by the recent advances in the microarray manufacturing) are determined. A collection of differentially expressed genes would therefore account, at the genomic/genetic level, for the distinctiveness of the two tissues, type of cells, or states. Certain multivariate distances are employed to evaluate such distinctiveness according to this invention.
For example, a probability distance and its nonparametric estimate may be used in this context. Let μ and ν be two probability measures defined on the Euclidean space. Let L(x,y) be a strictly negative definite kernel, that is

$${\sum }_{i , j = 1}^{s} L ( {x}_{i} , {x}_{j} ) {h}_{i} {h}_{j} \leq 0 for any {x}_{1} , … , {x}_{s} and {h}_{1} , … , {h}_{s} , \\ {\sum }_{i = 1}^{s} {h}_{i} = 0$$

with equality if and only if all hᵢ=0. It can be shown that a probability distance N(μ, ν) as defined below is a metric in the space of all probability measures on R^{d}.
N(μ,ν)=2∫_{R}_{^{d}}∫_{R}_{^{d}}L(x,y)dμ(x)dν(y)−∫_{R}_{^{d}}∫_{R}_{^{d}} L(x,y)dμ(x)dμ(y)−∫_{R}_{^{d}}∫_{R}_{^{d}}L(x,y)dν(x)dν(y)
Consider two independent samples, consisting of n₁ and n₂ observations respectively, represented by the d-dimensional vectors x₁, . . . ,xₙ₁ and y₁, . . . , yₙ₂. An empirical counterpart of N(μ, ν) may be represented as follows

$$\begin{matrix}\hat{N} = N ( {μ}_{n1} , {v}_{n2} ) \\ = \frac{1}{{n}_{1} {n}_{2}} {\sum }_{i = 1}^{n1} {\sum }_{j = 1}^{n2} 2 L ( {x}_{i} , {y}_{j} ) - \\ \frac{1}{{n}_{1}^{2}} {\sum }_{i = 1}^{n1} {\sum }_{j = 1}^{n1} L ( {x}_{i} , {x}_{j} ) - \frac{1}{{n}_{2}^{2}} {\sum }_{i = 1}^{n2} {\sum }_{j = 1}^{n2} L ( {y}_{i} , {y}_{j} ) .\end{matrix}$$

A pertinent kernel function L needs to be chosen when the probability distance N(μ, ν) is used. Appropriate choices include the Euclidean distance between ranks and a monotone function of the Euclidean distance satisfying the condition of negative definiteness. Additionally, an alternative class of kernel functions may be used to measure pairwise gene interaction.

Let x and y denote observations in two samples on a gene set and x^{r} and y^{r} denote the corresponding rank-adjusted observations. Consider either of these observations to be points in Euclidean space. Let S be a measurable subset of R^{d}. Define L_{S} by the rule L_{S}(x,y)=0 if both xεS and yεS and L_{S}(x,y)=1 otherwise. L_{S} is a negative definite kernel. Suppose, XᵢεS, 1≦i≦r, and xᵢ∉S, r+1≦i≦s, then one would have

$${\sum }_{i , j = 1}^{s} ( 1 - {L}_{S} ( {x}_{i} , {y}_{j} ) ) {h}_{i} {h}_{j} = {\sum }_{i , j = 1}^{r} {h}_{i} {h}_{j} = {( {\sum }_{i , j = 1}^{r} {h}_{i} )}^{2} \geq 0.$$

Thus (1−L_{S}) is a positive definite kernel.

More generally, let ƒ(x) be a function from a space to the interval [0,1], and define L_{ƒ}(x,y)=max(ƒ(x),ƒ(y)), then L_{ƒ} is a negative definite kernel. Also, if one defines g_{α}(x,y)=0 provides both ƒ(x)>α and ƒ(y)>α and g_{α}(x,y)=1 otherwise, then, from the previous paragraph, g_{α} is a negative definite kernel. It follows from the equality L_{ƒ}(x,y)=∫₀¹g_{α}(x,y)dα that L_{ƒ} is negative definite. Since a negative definite kernel is unaffected by an arbitrary additive shift, it is clear that L_{ƒ}(x,y)=max(ƒ(x),ƒ(y)) will be a negative definite kernel for any bounded function ƒ.

If wᵢ are positive weights and ƒᵢ, 1≦i≦d, are functions from to [0,1], then

$$L = {\sum }_{i = 1}^{d} {w}_{i} {L}_{{f}_{i}}$$

is also a negative definite kernel. From the foregoing derivations, one would also have: if {ƒᵢ} separates points, that is, ƒᵢ(x)=ƒᵢ(y) for all i implies x=y, then L is strictly negative definite.

Negative definite kernels of the type described above may be combined with the usual Euclidean distance to form composite kernel functions. For example, define a region function R_{q}(ν,ν)=q└qν┘+└qν┘ (here └·┘ denotes the floor function, its value is the largest integer not exceeding the argument and q≧2 is an integer parameter). This function is constant on each of the q² obtained by dividing the sides of the (0,1)² into q equal segments. Then the following kernels on the ranked data may be defined:

$${L}_{1} ( {x}^{r} , {y}^{r} ) = \sqrt{{\sum }_{g ∈ S} {( {x}_{g}^{r} - {y}_{g}^{r} )}^{2}} , \\ \begin{matrix}{L}_{2} ( {x}^{r} , {y}^{r} ) = {w}_{1} {L}_{1} ( {x}^{r} , {y}^{r} ) + \\ {w}_{2} {\sum }_{( {g}_{1} , {g}_{2} ) ∈ {S}^{2}} ( 1 - I \{ {R}_{q} ( {x}_{{g}_{1}}^{r} , {x}_{{g}_{2}}^{r} ) \\ = {R}_{q} ( {y}_{{g}_{1}}^{r} , {y}_{{g}_{2}}^{r} ) \} ) ,\end{matrix}$$

where I is the indicator function. Then L₁ is the standard Euclidean distance and L₂ falls into the class described above. We choose the weights w₁ and w₂ to balance the two components of L₂ with respect to their maximum values:

$${w}_{1} = 1 / {d}_{max} and {w}_{2} = 1 / ( \begin{matrix}{d}_{max} \\ 2\end{matrix} ) ,$$

where dₘₐₓ is the maximum subset dimension under consideration. The second component of the kernel will be insensitive to perturbation, yet pick up sets of genes that have similar expression levels across samples in one tissue and different expression patterns in the two tissues.

In another alternative embodiment, a function L_{ƒ} is based on the correlation coefficient. Let xⁿ and yⁿ denote normalized data such that the tissue-specific sample mean and variance are zero and one respectively. For each pair of genes g₁ and g₂, consider the function ƒ_{g}_{₁}_{,g}_{₂} (xⁿ)=x_{g1}ⁿx_{g2}ⁿ. The corresponding negative definite kernel L_{g1,g2} will detect differences in correlation between the two tissues. For example, if the expressions of g₁ and g₂ have correlation coefficient ρ in one tissue and are uncorrelated in the other, it follows from 2 max(ρ,0)−max(ρ,ρ)−max(0,0)=|p| that the corresponding distance between the tissues will be approximately equal to |ρ|.

A negative definite kernel may, in this embodiment, be defined as:

$${L}_{3} ( x , y ) = {w}_{1} {L}_{1} ( x , y ) + {w}_{2} {\sum }_{( {g}_{1} , {g}_{2} )} {L}_{{g}_{1} , {g}_{2}} ( x , y )$$

The weights w₁ and w₂ may be chosen to balance the contribution of the two components. A distance based on L₃ will tend to pick up sets of genes with separated means and differences in correlation in the two samples.

In various embodiments of this invention, once an aforementioned multivariate distance is selected, it may be used to search for a subset(s) of genes that are differentially expressed between the two tissues, types of cells, or biological states as the corresponding values of the distance are maximized. The size of such subsets is predetermined, which are typically small since they are limited by the available sample replicates. In theory, all subsets of a predetermined size need to be evaluated in terms of the adopted distance and the one that provides a maximum distance should be chosen as the final set of differentially expressed genes. In practice, however, the number of possible subsets exponentially increases with the total number of ge nes involved and, consequently, the exhaustive search procedures as well as the branch-and-bound method (see, e.g., Fukunaga K., (1990), Introduction to Statistical Pattern Recognition, Academic Press, London, 2nd.) become computationally prohibitive. Therefore, various stepwise random search procedures may be advantageously adopted according to this invention in identifying subsets of differentially expressed genes of a predetermined size.

In this connection, the search for a subset of genes with the best discrimination between two tissues, type of cells, or states often turns up overly-optimistic conclusions due to overfitting, i.e., finding overly specific patterns that do not extend to new samples. To mitigate such local selection bias, cross validation techniques may be adopted in random searches according to this invention, an example procedure (Procedure 1) is provided as follows:

1. Randomly divide the data into v groups of an approximate equal size;

2. Drop one of the n groups and find the optimal subset of genes using only the data from v−1 group, based on the evaluation of the applicable probability distance.

3. Repeat step 2 in succession for each of the groups, obtaining v optimal subsets.

4. Combine these sets by selecting the genes with the highest frequencies of occurrences.

In alternative embodiments, multiple local searches may be performed and then the resulting locally sub-optimal subsets may be integrated such that a final set of differentially expressed genes may be identified (e.g., by including the genes with the highest frequency of occurrences in the locally sub-optimal subsets).

Establishing Larger Sets of Genes Based on the Identified Smaller Subsets

As discussed above, random search procedures based on certain probability distances may be utilized to identify a subset of differentially expressed genes of a predetermined size. And, since a predetermined size as such often is limited by the scarcity of the sample size (especially when the total number of genes is large and the dimensionality of the microarray data is high), it is desirable to find a way to enlarge the size of the set of differentially expressed genes identified.

In one embodiment of this invention, a successive selection procedure is adopted to eliminate groups of genes after each run of the random search procedure, until no more subsets of genes can be found that satisfy the search criteria. The final set of differentially expressed genes would then include all the removed genes at each step. Essential to this method is the formulation of a stopping rule at each step.

The formulation of such an appropriate stopping rule turns on the evaluation of the properties of an optimal set of genes in a “no-difference” data set. Various quality functions may be used in this context to provide a model to evaluate such properties. For example, certain multivariate distances are used as the quality function in various embodiment of this invention. The selection process based on the application of such multivariate quality functions would necessarily be influenced by the covariance structure of the microarray data. Thus, the “no-difference” baseline data (i.e., corresponding to the null-distribution) ought to be generated in such a way that the covariance data structure is preserved. The following two-step “resampling” process (Procedure 2) meets such requirement. The first step ensures that the marginal means of the two data sets (may have been obtained from two tissues, types of cells, or biological states) have the same true mean. And, the second step mimics the biological variability through permutation.

Denote the adjusted fluorescence level for gene i, i=1 . . . , p in the two tissues by Xᵢⱼ, j=1 . . . , n₁ and Yᵢⱼ, j=1 . . . , n₂, respectively.

1. For each gene i, i=1, . . . ,p shift the values from the two data sets so they are centered at the overall mean for this gene, that is

$${X}_{ij}^{*} = {X}_{ij} - {\overline{X}}_{i} . + \frac{{n}_{1} {\overline{X}}_{i} . + {n}_{2} {\overline{Y}}_{i} .}{{n}_{1} + {n}_{2}} , {Y}_{ij}^{*} = {Y}_{ij} - {\overline{Y}}_{i} . + \frac{{n}_{1} {\overline{X}}_{i} . + {n}_{2} {\overline{Y}}_{i} .}{{n}_{1} + {n}_{2}}$$

2. Randomly permute the resulting n₁+n₂ vectors. The first n₁ and the last n₂ vectors provide a random sample from the null-distribution.

Based on this permutation resampling scheme, the null-distributions of various quantitative characteristics of the optimal gene set may be estimated. For example, the associated probability distance, cross validation classification rate (using a selected subset upon cross validation), and test set classification rate (using an independent test set) may be considered. A test set classification rate is calculated by classifying each sample from an independent test set using the selected subset of genes and the entire training set and determining the rate of the correct classification. A cross-validation classification rate is calculated by classifying each sample in the training set (in the absence of a test set) using the selected subset of genes and the rest of the training set and determining the rate of the correct classification. Generally, the test set classification rate may be most desirable but, due to the scarcity of samples, an appropriate test set is often unavailable. In such situations, the between-tissue distance associated with gene sets may be a good and stable proxy for the classification rate.

According to a particular embodiment, a probability distance-based successive-selection procedure is adopted in selecting a subset of genes that are differentially expressed in two tissues, type of cells, or biological states, as outlined below (Procedure 3). The successive selection based on cross-validation or test set classification rates may be similarly adopted in connection with random searches in alternative embodiments of this invention.

The following procedure (Procedure 3) starts with the selection of a subset of genes with a size k and requires a significance level a for defining a percentile of the null-distribution of the data sets.

1. Form m independent permutation samples of sizes n1 and n2, respectively, from n1+n2 observations (arrays/slides). For each of the m permutation samples, find an optimal k-element subset of genes for which the associated probability distance attains its maximum. Estimate from the permutation samples the top α^{th} percentile D_{α} of the baseline distribution of the optimal distance (referred to as the null-distribution).

2. Returning to the original two data set setting, find the k-element optimal set of genes for which the associated probability distance attains its maximum and denote it by G₁. If the associated probability distance D(G₁)>D_{α}, then continue, otherwise stop the search.

3. In the t^{th} iteration, discard sets G₁, . . . , Gₜ₋₁ and find the k-element optimal set Gₜ from the remaining genes. If the associated distance D(Gₜ)>D_{α}, then continue with this step (next iteration), otherwise proceed to step 4.

4. The final set of differentially expressed genes are defined by the union ∪ⱼ₌₁^{t}Gⱼ.

Computer Simulation of the Improved Random Search

A simulation study was performed to evaluate the improved random search method with the successive elimination procedure. A total of 1000 genes was divided into subsets of equal size 20. In the first data set, no differential expression was imposed, and hence any difference shown would be due to the within-tissue “biological variability.” In the second data set, one of the subsets (including 20 mutually dependent expression signals) was set to be differentially expressed with a ratio of two. The correlation structure was kept the same in the two data sets. Further, an independent test set of 100 observations (with equal proportions of the two hypothetical tissues) was simulated for the two data sets in order to estimate the true classification rate of the selected gene sets.

A cross-validated random search was performed in accordance with Procedure 1 supra. Particularly, step 2 of Procedure 1 was carried out in the following details (Procedure 4):

1. Randomly select k genes to form the initial approximation; calculate the associated probability distance between the two data sets for this subset of genes.

2. Replace at random one gene from the current subset with a gene outside of the subset and calculate the value of the associated probability distance for the resulting new subset; if the distance is larger than that of the previous subset, keep the new subset and, otherwise, revert to the previous subset.

3. Repeat the process until a predetermined number M of iterations is reached.

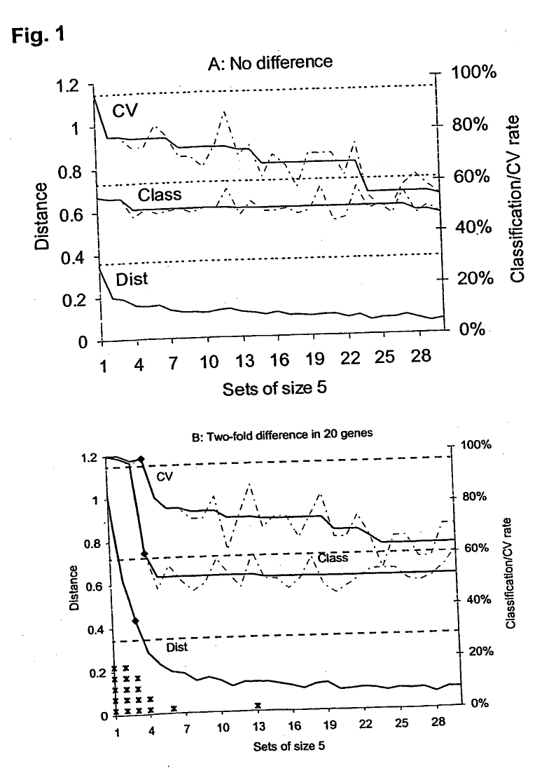

In this particular simulation, M was set at 100,000. The successive search for 5-member optimal gene sets (k=5) was performed using the 10-fold (v=10) cross-validated search procedure (Procedure 1). The Euclidean distance was chosen for the kernel L(x, y) in the distance measure. For each of the successive optimal sets Gᵢ, the corresponding optimal distance was recorded and the tissue classification rate was estimated using both cross validation (using the selected gene set) and the independent test set. The results are shown in FIG. 1.

Referring to FIG. 1, the top panel shows the results for the data set that had no difference imposed whereas the bottom panel shows the results for the is data set that had a subset of 20 genes to be differentially expressed in the two hypothetical tissues. In both panels, the left y axis represents the associated probability distance while the right y axis denotes the classification rate based on the independent test set (hence test set classification rate—“Class”) and the classification rate based on cross validation using selected gene set (hence cross validation classification rate—“CV”). The x axis of both panel denotes the number of subsets of genes with a predetermined size of 5. As shown in both panels of FIG. 1, the estimate of the test set classification rate and that of the cross validation classification rate are both highly variable for both data sets, whereas the associated distance (Dist) is decreasing monotonically. Since the optimal sets were selected based on the associated probability distance in this simulation, the observed monotonicity confirms the ability of the random search procedure of this invention to find an optimal subset.

To reduce the observed variability of the classification rate estimates, isotonic regression (see, Robertson T. et al., (1988) Order Restricted Statistical Inference, Wiley, London) was performed to smooth the corresponding curves and thereby generate the corresponding solid lines in FIG. 1, assuming the true rates to be non-increasing. The dotted horizontal lines represent the level of the 99th percentile of the null-distributions of the corresponding measures (i.e., the associated probability distance, the test set classification rate, and the cross validation classification rate); they were estimated by generating 100 random permutation samples that mimic “no-difference” data in accordance with Procedure 2 supra. For the first data set, referring to the top panel of FIG. 1, all the observed curves lie entirely below their cutoff values, which demonstrates that the random search of the invention with the successive elimination procedure correctly identifies no differentially expressed genes in the first data set.

For the second data set, since 20 genes were set to be differentially expressed, the selection should stop after 4 iterations (i.e., to identify 4 subsets of 5 genes). Referring to the bottom panel of FIG. 1, the distance curve (Dist) passes its cutoff level after the third iteration, whereas the cross validation and the test set classification curves pass their cutoff levels after the fourth iteration. Thus, in the simulated random search, the successive elimination procedures based on associated distance, the test set classification rate, as well as the cross validation classification rate all performed satisfactorily in this simulation, with the distance-based procedure slightly inferior to the other two as it stopped early. However, the distance-based procedure demonstrated superior stability and therefore it remains a powerful alternative in certain embodiments of this invention. In summary, the distance based cutoff identified 14/20=70% of the 20 differentially expressed genes with a false positive (type I error) rate of only 1/15=6.7%, while the two classification based cutoffs identified 16/20=80% of the differentially expressed genes with a 4/20=20% false positive rate. The differentially expressed genes were marked with stars in the bottom panel of FIG. 1.

The invention is further described by the following examples, which are illustrative of the invention but do not limit the invention in any manner.

### EXAMPLE 1

### A Source Code Segment Implementing Successive Selection and Re-Sampling

```
unit FExclude;
interface
uses
  Windows, Messages, SysUtils, Classes, Graphics, Controls, Forms,
  Dialogs, Spin, StdCtrls, Grids, Aligrid, EnhCBox, NumIO,
  ComCtrls, Matrix, Vector;
type
  TExcludeForm = class(TForm)
    NExcludeSteps: TSpinEdit;
    Label1: TLabel;
    RunEliminationButton: TButton;
    ExcludeResult: TStringAlignGrid;
    SaveButton: TButton;
    SaveDialog: TSaveDialog;
    Label2: TLabel;
    Class1Box: TEnhComboBox;
    Class2Box: TEnhComboBox;
    H0Button: TButton;
    H0repInput: TNumIO;
    Label3: TLabel;
    ExcludePB: TProgressBar;
    RandomClusterButton: TButton;
    RunFromDiffClButton: TButton;
    procedure RunEliminationButtonClick(Sender: TObject);
    procedure FormCreate(Sender: TObject);
    procedure SaveButtonClick(Sender: TObject);
    procedure ExcludeResultKeyDown(Sender: TObject; var Key: Word;
      Shift: TShiftState);
    procedure ExcludeResultFixedRowClick(Sender: TObject; row:
    Integer);
    procedure ClassBoxDblClick(Sender: TObject);
    procedure H0ButtonClick(Sender: TObject);
    procedure RandomClusterButtonClick(Sender: TObject);
    procedure RunFromDiffClButtonClick(Sender: TObject);
  private
    { Private declarations }
    procedure OneEliminationStep;
    function ClassifyTestSets(filename: string; normal, centerdata:
    boolean)
        : string;
  public
    { Public declarations }
    PermMat1, PermMat2: TMatrix;
    RandDistCurves, ClassCurves, RandCV: TMatrix;
    MeanDiff: TVector;
  end;
var
  ExcludeForm: TExcludeForm;
implementation
uses FDiffClust, DiffCluster, ClassificationF, readdata, RandomGen;
{$R *.DFM}
procedure TExcludeForm.RunEliminationButtonClick(Sender: TObject);
var i, nsteps: integer;
  testset: boolean;
begin
  nsteps:= NExcludeSteps.Value;
  ExcludeResult.RowCount:= nsteps+1;
  testset:= (FileExists(Class1Box.Text)) and
          (FileExists(Class2Box.Text));
  ExcludePB.Position:= 0;
  ExcludePB.max:= nsteps + 1;
  for i:= 1 to nsteps do begin
    try
      ExcludePB.StepIt;
      ExcludePB.Update;
      BatchProcess:= True;
      DiffClusterForm.FindDiffClusterButtonClick(self);
      DiffClusterForm.DistButtonClick(self);
      if not Assigned(ClassificationForm) then
        ClassificationForm:= TClassificationForm.Create(self);
      if DiffClusterForm.AdjustType.ItemIndex <=2 then
        ClassificationForm.AdjustOptions.ItemIndex:=
          DiffClusterForm.AdjustType.ItemIndex;
      ClassificationForm.CrossvalidButtonClick(self);
      ExcludeResult.Row:= i;
      ExcludeResult.CellAsInt[0,i]:= i;
      ExcludeResult.Cells[1,i]:= DiffClusterForm.OutputMemo.Text;
      ExcludeResult.Cells[2,i]:= ClassificationForm.
      PCorrectOutput.Text+‘%’;
      ExcludeResult.Cells[3,i]:= DiffClusterForm.DistOutput.Caption;
      ExcludeResult.Cells[4,i]:=
        FloatToStrF(MinFreqInDiffcl*100,ffFixed,3,1)+‘%’;
      ExcludeResult.Cells[5,i]:=
        FloatToStrF(MaxFreqInDiffcl*100,ffFixed,3,1)+‘%’;
      if testset then begin
        ExcludeResultCells[6,i]:=
          ClassifyTestSets(Class1Box.Text,True,False)+‘%’;
        ExcludeResult.Cells[7,i]:=
          ClassifyTestSets(Class2Box.Text,False,False)+‘%’;
      end;
      OneEliminationStep;
    finally
      BatchProcess:= False;
      end;
    end;
    ExcludePB.Position:= 0;
  end;
  procedure TExcludeForm.OneEliminationStep;
  var i, gene: integer;
    genelist:= TStringList;
  begin
    genelist: TStringList.Create;
    try
      genelist.CommaText:= DiffClusterForm.OutputMemo.Text;
      for i:= 0 to genelist.Count−1 do begin
      gene:= StrToInt(genelist[i]);
      UseGeneInd[gene−1]:= 0;
    end;
  finally
    genelist.Free;
  end;
end;
function TExcludeForm.ClassifyTestSets;
begin
  with ClassificationForm do begin
    ClassifFileName.Text:= filename;
    if centerdata then
      RunButtonClick(H0Button)
    else
      RunButtonClick(RunEliminationButton);
    if normal then
      ActualClassOptions.ItemIndex:= 0
    else
      ActualClassOptions.ItemIndex:= 1;
    Result:= PCorrectOutput.Text;
  end;
end;
procedure TExcludeForm.FormCreate(Sender: TObject);
begin
    ExcludeResult.AllowCutnPaste:= True;
    ExcludeResult.PasteEditableOnly:= False;
end;
procedure TExcludeForm.SaveButtonClick(Sender: TObject);
begin
  if SaveDialog.Execute then
    ExcludeResult.SaveToFile(SaveDialog.FileName);
end;
procedure TExcludeForm.ExcludeResultKeyDown(Sender: TObject;
var Key: Word;
  Shift: TShiftState);
begin
    if (Key=67) then
      if (Shift=[ssCtrl]) then
      with ExcludeResult do
      Contents2CSVClipboard(#9,Selection);
end;
procedure TExcludeForm.ExcludeResultFixedRowClick(Sender: TObject;
  row: Integer);
begin
    if ExcludeResult.Cells[1,row]<>” then
      with DiffClusterForm do begin
      OutputMemo.Clear;
      OutputMemo.Text:=ExcludeResult. Cells[1,row];
      end;
end;
procedure TExcludeForm.ClassBoxDblClick(Sender: TObject);
begin
    if SaveDialog.Execute then begin
      (Sender as TEnhComboBox).Text= SaveDialog.FileName;
    end;
end;
procedure TExcludeForm.H0ButtonClick(Sender: TObject);
var A, B: TMatrix;
  ss1, ss2, s, i, j, k, nH0rep, nsteps: integer;
  sampleperm: array of double;
  stepmin, stepmax: double;
  testset: boolean;
begin
  case DiffClusterForm.AdjustType.ItemIndex of
    0: begin A:= normal; B:= polyp end;
    1: begin A:= renormal; B:= repolyp end;
    2: begin A:= rnormal; B:= rpolyp end;
    else Exit;
  end;
  testset:= (FileExists(Class1Box.Text)) and
      (FileExists(Class2Box.Text));
  ss1:= A.NrOfRows;
  ss2:= B.NrOfRows;
  SetLength(sampleperm, ss1+ss2);
  for i:= 1 to ss1 do
    sampleperm[i−1]:= i;
  for i:= 1 to ss2 do
    sampleperm[ss1+i−1]:= ss1+i;
  PermMat1:= TMatrix.Create(A.NrOfColumns, ss1);
  PermMat2:= TMatrix.Create(B.NrOfColumns, ss2);
  nH0rep:= Trunc(H0repInput.Value);
  nsteps:= NExcludeSteps.Value;
  RandDistCurves:= TMatrix.Create(nH0rep, nsteps);
  RandCV:= TMatrix.Create(nH0rep, nsteps);
  if not Assigned(ClassificationForm) then
    ClassificationForm:= TClassificationForm.Create(self);
  if testset then begin
    ClassCurves:= TMatrix.Create(nH0rep, nsteps);
  end;
  //calculate vector of gene-mean differences
  MeanDiff:= TVector.Create(lines);
  for i:= 1 to lines do
    MeanDiff[i]:= B.Sum(i,1,i,ss2)/ss2−A.Sum(i,1,i,ss1)/ss1;
  BatchProcess:= True;
  ExcludePB.Position:= 0;
  ExcludePB.Max:= nH0rep+1;
  try
    for i:= 1 to nH0rep do begin
    ExcludePB.StepIt;
    //include all genes
    for j:= 0 to high(UseGeneInd) do
    UseGeneInd[j]:= 1;
    //setup randomly permuted samples
    RandomPerm(sampleperm);
    // bootstrap samples
    { for j:= 0 to ss1+ss2−1 do
    sampleperm[j]:= Ran0(ss1+ss2−1)+1; }
    for j:= 0 to ss1−1 do
    if sampleperm[j]>ss1 then begin
      s:= Trunc(sampleperm[j])−ss1;
      for k:= 1 to lines do
      PermMat1[k,j+1]:= B[k,s]−ss1/(ss1+ss2)*MeanDiff[k];
    end
    else begin
      s:= Trunc(sampleperm[j]);
      for k:= 1 to lines do
      PermMat1[k,j+1]:= A[k,s]+ss2/(ss1+ss2)*MeanDiff[k];
    end;
for j:= ss1 to ss1+ss2−1 do
  if sampleperm[j]>ss1 then begin
    s:= Trunc(sampleperm[j])−ss1;
    for k:= 1 to lines do
      PermMat2[k,j−ss1+1]:= B[k,s]−ss1/(ss1+ss2)*MeanDiff[k];
  end
  else begin
    s:= Trunc(sampleperm[j]);
    for k:= 1 to lines do
      PermMat2[k,j−ss1+1]:= A[k,s]+ss2/(ss1+ss2)*MeanDiff[k];
  end;
if i=1 then begin
  PermMat1.StoreOnFile(1,1,100,ss1,‘perm1.txt’);
  PermMat2.StoreOnFile(1,1,100,ss2,‘perm2.txt’);
end;
//calculate distance curve for this permutation
  for j:= 1 to nsteps do begin
  DiffClusterForm.FindDiffClusterButtonClick(H0Button);
  DiffClusterForm.DistButtonClick(H0Button);
  RandDistCurves[i,j]:= StrToFloat(DiffClusterForm.
  DistOutput.Caption);
  if DiffClusterForm.AdjustType.ItemIndex <=2 then
    ClassificationForm.AdjustOptions.ItemIndex:=
      DiffClusterForm.AdjustType.ItemIndex;
  ClassificationForm.CrossvalidButtonClick(H0Button);
  RandCV[i,j]:= StrToFloat(ClassificationForm.PCorrectOutput.Text);
  if testset then begin
    ClassCurves[i,j]:=
      (StrToFloat(ClassifyTestSets(Class1Box.Text,True,True)) +
        StrToFloat(ClassifyTestSets(Class2Box.Text,False,True)))/2;
    if ClassCurves[i,j]=100 then
      ShowMessage(‘Perfect again:(’);
  end;
  OneEliminationStep;
  end;
end;
{ //calculate percentiles (min-max)
  RandDistCurves.Resize(nH0rep+2,nsteps);
  for j:= 1 to nsteps do begin
  RandDistCurves.MinMax(1,j,nH0rep,j,stepmin,stepmax);
  RandDistCurves[nH0rep+1,j]:= stepmin;
  RandDistCurves[nH0rep+2,j]:= stepmax;
  end; }
finally
  PermMat1.Free;
  PermMat2.Free;
  RandDistCurves.StoreOnFile(1, 1, nH0rep, nsteps, ‘randdistcurves.txt’);
  RandDistCurves.Free;
  RandCV.StoreOnFile(1, 1, nH0rep, nsteps, ‘randCV.txt’);
  RandCV.Free;
  MeanDiff.Free;
  if testset then begin
  ClassCurves.StoreOnFile(1, 1, nH0rep, nsteps, ‘classcurves.txt’);
  ClassCurves.Free;
  end;
  ExcludePB.Position:= 0;
  //include all genes
  for i:= 0 to high(UseGeneInd) do
    UseGeneInd[i]:= 1;
  BatchProcess:= False;
  end;
end;
procedure TExcludeForm.RandomClusterButtonClick(Sender: TObject);
var nH0rep, i, j, size: integer;
  RandDist, RandClass: TVector;
  testset: boolean;
  indexarr: array of double,
  F: TextFile;
begin
  nH0rep:= Trunc(H0repInput.Value);
  size:= Trunc(DiffClusterForm.NumElemInput.Value);
  initprogress(ExcludePB, nH0rep);
  testset:= (FileExists(Class1Box.Text)) and
        (FileExists(Class2Box.Text));
  RandDist:= TVector.Create(nH0rep);
  if testset then begin
    RandClass:= TVector.Create(nH0rep);
    if not Assigned(ClassificationForm) then
      ClassificationForm:= TClassificationForm.Create(self);
  end;
  SetLength(indexarr, lines);
  for i:= 0 to lines−1 do
    indexarr[i]:= i+1;
  BatchProcess:= True;
  try
    for i:= 1 to nH0rep do begin
      RandomPerm(indexarr);
      with DiffClusterForm do begin
      OutputMemo.Clear;
      OutputMemo.Text:= IntToStr(Trunc(indexarr[0]));
      for j:= 2 to size do
        OutputMemo.Text:= OutputMemo.Text+ ‘, ’ +
            IntToStr(Trunc(indexarr[j]));
      DistButtonClick(RandomClusterButton);
      RandDist[i]:= StrToFloat(DistOutput.Caption);
      if testset then begin
        RandClass[i]:=
          (StrToFloat(ClassifyTestSets(Class1Box.Text,True,True)) +
          StrToFloat(ClassifyTestSets(Class2Box.Text,False,True)))/2;
      end;
      end;
      stepup(ExcludePB);
    end;
    finally
      BatchProcess:= False;
      initprogress(ExcludePB, 1);
      AssignFile(F,‘randdist_class.txt’);
      Rewrite(F);
      if testset then begin
      Writeln(F, ‘distance’, chr(9), ‘aveclass’);
      for i:= 1 to nH0rep do
        writeln(F, RandDist[i], chr(9), RandClass[i]);
      CloseFile(F);
      RandDist.Free;
      RandClass.Free;
  end
  else begin
    Writeln(F, ‘distance’);
    for i:= 1 to nH0rep do
      writeln(F, RandDist[i]);
    CloseFile(F);
    RandDist.Free;
    end;
  end;
end;
procedure TExcludeForm.RunFromDiffClButtonClick(Sender: TObject);
var GeneList: TStringList;
  i, j, nsteps, nelem, 1length: integer;
  testset: boolean;
begin
  GeneList:= TStringList.Create;
  GeneList.CommaText:= DiffClusterForm.OutputMemo.Text;
  1length:= GeneList.Count;
  nelem:= Trunc(DiffClusterForm.NumElemInput.Value);
  nsteps:= Trunc(1length/nelem);
  ExcludeResult.RowCount:= nsteps+1;
  testset:= (FileExists(Class1Box.Text)) and
        (FileExists(Class2Box.Text));
  ExcludePB.Position:= 0;
  ExcludePB.max:= nsteps + 1;
  for i:= 1 to nsteps do begin
    try
    ExcludePB.StepIt;
    ExcludePB.Update;
    BatchProcess:= True;
    with DiffClusterForm.OutputMemo do begin
      Clear;
      Text:= GeneList[(i−1)*nelem];
      for j:= 1 to nelem−1 do
        Text:= Text + ‘, ’ + GeneList[(i−1)*nelem + j];
    end;
    DiffClusterForm.DistButtonClick(self);
    if not Assigned(ClassificationForm) then
      ClassificationForm:= TClassificationForm.Create(self);
    if DiffClusterForm.AdjustType.ItemIndex <=2 then
      ClassificationForm.AdjustOptions.ItemIndex:=
      DiffClusterForm.AdjustType.ItemIndex;
    ClassificationForm.CrossvalidButtonClick(self);
    ExcludeResult.Row:= i;
    ExcludeResult.CellAsInt[0,i]:= i;
    ExcludeResult.Cells[1,i]:= DiffClusterForm.OutputMemo.Text;
    ExcludeResult.Cells[2,i]:= ClassificationForm.PCorrectOutput.
    Text+‘%’;
    ExcludeResult.Cells[3,i]:= DiffClusterForm.DistOutput.Caption;
    ExcludeResult.Cells[4,i]:=
      FloatToStrF(MinFreqInDiffcl*100,ffFixed,3,1)+‘%’;
    ExcludeResult.Cells[5,i]:=
      FloatToStrF(MaxFreqInDiffcl*100,ffFixed,3,1)+‘%’;
    if testset then begin
      ExcludeResult.Cells[6,i]:=
      ClassifyTestSets(Class1Box.Text,True,False)+‘%’;
      ExcludeResult.Cells[7,i]:=
      ClassifyTestSets(Class2Box.Text,False,False)+‘%’;
    end;
    finally
    BatchProcess:= False;
    end;
  end;
  ExcludePB.Position:= 0;
  DiffClusterForm.OutputMemo.Text:= GeneList.CommaText
  GeneList.Free;
end;
end.
```

### EXAMPLE 2

### Analysis on Microarray Expression Data from Colon Cancer Cell Lines

Two colon cancer cell lines were used in this experiment. HT29 cells represent advanced, highly aggressive colon tumors. They contain mutations in both the APC gene and p53 gene, two tumor suppressor genes that frequently mutate during colon tumorigenesis. HCT116 cells manifest less aggressive colon tumors and harbor functional p53 and APC. They are defective in DNA repair. The experiment was performed with three RNA samples (1 μg RNA each). Cy-3-dCTP (green) was used to label HCT116 cells while Cy-5-dCTP (red) was used for HT29 cells. Six independent replicates were obtained each for HT29 and HCT116 cell lines. In addition, the data from a separate experiment was used as the independent test set, which contained eight replicates for each cell line.

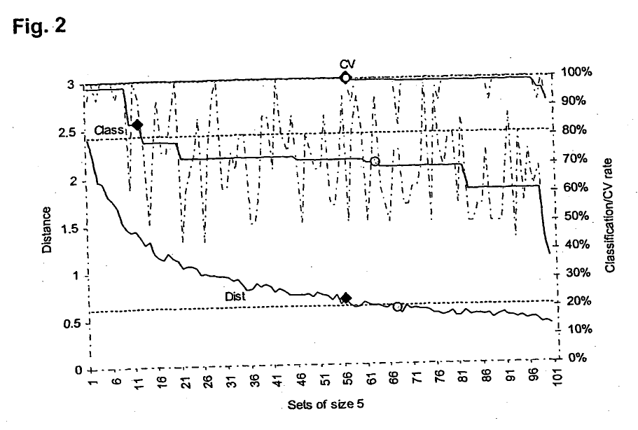

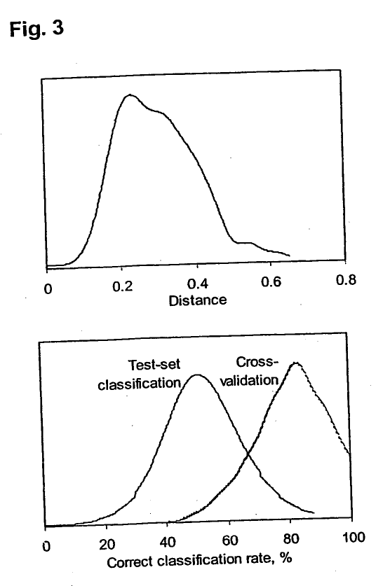

The analysis of differential expression of the two cell lines was carried out similarly as the computer simulation study described supra. The number of permutation was set at 300 in this analysis (in accordance with Procedure 2 supra), and the size of the subsets is k=5 (in accordance with Procedure 4 supra). The results from application of the three stopping rules (the associated probability distance, the test set classification rate, and the cross validation classification rate) are shown in FIG. 2. The estimates of the corresponding null-distributions are shown in FIG. 3.

Referring to FIG. 2, the left y axis represents the associated probability distance while the right y axis denotes the classification rate based on the independent test set (hence test set classification rate—“Class”) and the classification rate based on cross validation using selected gene set (hence cross validation classification rate—“CV”). The x axis denotes the number of subsets of genes with a predetermined size of 5. The dotted horizontal lines represent the level of the 99th percentile of the null-distributions of the corresponding measures (i.e., the associated probability distance, the test set classification rate, and the cross validation classification rate); they were estimated by generating 300 random permutation samples that mimic “no-difference” data in accordance with Procedure 2 supra. Using the 99th percentile of the null-distribution as the cutoff, the cross validation rate approach stops at the 57th subset and the distance-based criteria stops at 56th subset (referring to the black diamonds on the solid lines “CV” and “Dist” in FIG. 2). Whereas, the smoothed (via isotonic regression as discussed supra) test set classification rate drops below the cutoff much earlier, at the 12th subset (referring to the black diamond on the solid line “Class” in FIG. 2). However, is when the 95th percentile was used, the stopping points for all three measures were at closer vicinity relative to one other. The extremely high variability of the test set classification rate may be responsible for such discrepancy, since the test set data was generated in a separate and earlier experiment.

Further comparison was carried out between the multivariate random search procedure of this invention and a univariate selection approach. The genes were sorted according to the values of the corresponding marginal t-statistics and the top 56×5=280 genes were selected, such that the number of selected genes was identical to that identified by the multivariate distance-based cutoff criterion as discussed above. It was observed that the resulting two groups of differentially expressed genes share only 94 genes (33%). The first gene that did not appear in the group selected using the univariate approach—Hs.2867, Interferon-alpha induced 11.5 KD protein—appeared in the fourth subset G₄ of genes identified by the multivariate selection approach. Interestingly, another copy of the same gene did appear in both groups and that corresponding changes in the interferon pathway in the HT29 cell line are well known. Therefore, identification of this gene as being differentially expressed was an accurate conclusion and, the multivariate search method of this invention was advantageously more sensitive compared to the univariate approach.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

1. A method for identifying a set of genes from a multiplicity of genes whose expression levels at a first state and a second state are measured in replicates using one or more nucleotide arrays, thereby generating a first plurality of independent measurements of the expression levels for said first state and a second plurality of independent measurements of the expression levels for said second state, which method comprises the following sequential steps:
(a) identifying a quality function capable of evaluating the distinctiveness between the first plurality and the second plurality;
(b) forming a first predetermined number of permutations from the first and the second pluralities, dividing said permutations into a first permutated plurality and a second permutated plurality, corresponding in size, to said first and second plurality, respectively, and identifying groups of genes the size of which is a second predetermined number, wherein the values of the quality function for the group of genes in said first permutated and second permutated pluralities attain the maximum;
(c) determining, from said first and second permutated pluralities, the top α^{th} percentile of the null distribution based on a quantitative characteristic of said groups of genes;
(d) identifying, based on the first and second pluralities, a subset of genes the size of which is said second predetermined number, wherein the values of the quality function for said subset of genes in said first and second pluralities attain the maximum;
(e) adding to the set of genes, said subset, if the value of said quantitative characteristic associated with said subset exceeds said top α^{th} percentile of the null distribution; and
(f) removing from the first and second pluralities, all measurements on said subset, if the maximum value of the quality function associated with said subset exceeds said top α^{th} percentile of the null distribution, and repeating steps (d)-(f) until no more measurements are left in the first and second pluralities or the value of said quantitative characteristic associated with the subset does not exceed said top α^{th} percentile of the null distribution.

2. The method of claim 1, wherein said states are selected from the group consisting of biological states, physiological states, pathological states, and prognostic states.

3. A method for identifying a set of genes from a multiplicity of genes whose expression levels in a first tissue and a second tissue are measured in replicates using one or more nucleotide arrays, thereby generating a first plurality of independent measurements of the expression levels for said first tissue and a second plurality of independent measurements of the expression levels for said second tissue, which method comprises:
(a) identifying a quality function capable of evaluating the distinctiveness between the first plurality and the second plurality;
(b) forming a first predetermined number of permutations from the first and the second pluralities, dividing said permutations into a first permutated plurality and a second permutated plurality, corresponding in size to said first and second plurality, respectively, and identifying groups of genes the size of which is a second predetermined number, wherein the values of the quality function for the group of genes in said first permutated and second permutated pluralities attain the maximum;
(c) determining, from said first and second permutated pluralities, the top α^{th} percentile of the null distribution based on a quantitative characteristic of said groups of genes;
(d) identifying, based on the first and second pluralities, a subset of genes the size of which is said second predetermined number, wherein the values of the quality function for said subset of genes in said first and second pluralities attain the maximum;
(e) adding to the set of genes, said subset, if the value of said quantitative characteristic associated with said subset exceeds said top α^{th} percentile of the null distribution; and
(f) removing from the first and second pluralities, all measurements on said subset, if the maximum value of the quality function associated with said subset exceeds said top α^{th} percentile of the null distribution, and repeating steps (d)-(f) until no more measurements are left in the first and second pluralities or the value of said quantitative characteristic associated with the subset does not exceed said top α^{th} percentile of the null distribution.

4. The method of claim 3, wherein said tissues are selected from the group consisting of normal lung tissues, cancer lung tissues, normal heart tissues, pathological heart tissues, normal and abnormal colon tissues, normal and abnormal renal tissues, normal and abnormal prostate tissues, and normal and abnormal breast tissues.

5. A method for identifying a set of genes from a multiplicity of genes whose expression levels in a first type of cells and a second type of cells are measured in replicates using one or more nucleotide arrays, thereby generating a first plurality of independent measurements of the expression levels for said first type of cells and a second plurality of independent measurements of the expression levels for said second types of cells, which method comprises:
(a) identifying a quality function capable of evaluating the distinctiveness between the first plurality and the second plurality;
(b) forming a first predetermined number of permutations from the first and the second pluralities, dividing said permutations into a first permutated plurality and a second permutated plurality, corresponding in size, to said first and second plurality, respectively, and identifying groups of genes the size of which is a second predetermined number, wherein the values of the quality function for the group of genes in said first permutated and second permutated pluralities attain the maximum;
(c) determining, from said first and second permutated pluralities, the top α^{th} percentile of the null distribution based on a quantitative characteristic of said groups of genes;
(d) identifying, based on the first and second pluralities, a subset of genes the size of which is said second predetermined number, wherein the values of the quality function for said subset of genes in said first and second pluralities attain the maximum;
(e) adding to the set of genes, said subset, if the value of said quantitative characteristic associated with said subset exceeds said top α^{th} percentile of the null distribution; and
(f) removing from the first and second pluralities, all measurements on said subset, if the maximum value of the quality function associated with said subset exceeds said top α^{th} percentile of the null distribution, and repeating steps (d)-(f) until no more measurements are left in the first and second pluralities or the value of said quantitative characteristic associated with the subset does not exceed said top α^{th} percentile of the null distribution.

6. The method of claim 5, wherein said types of cells are selected from the group consisting of normal lung cells, cancer lung cells, normal heart cells, pathological heart cells, normal and abnormal colon cells, normal and abnormal renal cells, normal and abnormal prostate cells, and normal and abnormal breast cells.

7. The method of claim 5, wherein said type of cells are selected from the group consisting of cultured cells and cells isolated from an organism.

8. The method of claim 1, 3, or 5, wherein said quality function is a probability distance function.

9. The method of claim 8, wherein said probability distance function is selected from the group consisting of the Mahalanobis distance and the Bhattacharya distance.

10. The method of claim 8, wherein the probability distance function is defined as:
N(μ,ν)=2∫_{R}_{^{d}}∫_{R}_{^{d}}L(x,y)dμ(x)dν(y)−∫_{R}_{^{d}}∫_{R}_{^{d}}L(x,y)dμ(x)dμ(y)−∫_{R}_{^{d}}∫_{R}_{^{d}}L(x,y)dν(x)dν(y)
where μ and ν are two probability measures defined on the Euclidean space, and L(x,y) is a strictly negative definite kernel.

11. The method of claim 10, wherein the negative definite kernel is combined with the Euclidean distance between x and y to form a composite kernel function.

12. The method of claim 1, 3, or 5, wherein the quantitative characteristic is selected from the group consisting of an associated probability distance, a test set classification rate, and a cross validation classification rate.

13. The method of claim 1, 3, or 5, wherein the formation of the permutations further comprises:
(i) shifting the measurements in the first and second pluralities such that the marginal means thereof share the same true mean; and
(ii) randomly permuting the resulting shifted measurements thereby forming a null-distribution of permutations.

14. The method of claim 1, 3, or 5, wherein the identifying further comprises:
(i) calculating the values of the quality function for said subset of genes in said first and second pluralities thereby evaluating the distinctiveness of said first and second pluralities;
(ii) substituting a gene in said subset with one outside of said subset, thereby generating a new subset, and repeating step (i), keeping the new subset if the distinctiveness increases and the original subset if otherwise; and
(iii) repeating steps (i) and (ii) for a fourth predetermined number of times.

15. The method of claim 1, 3, or 5, wherein the identifying further comprises:
(i) randomly dividing the first and the second pluralities into v groups of an approximate equal size;
(ii) removing one of said v groups from said first and second pluralities and identifying, from the resulting reduced first and second pluralities, a subset of genes for which the value of said quality function attains the maximum; and
(iii) repeating step (ii) for each of said v groups thereby obtaining v subsets of genes.

16. The method of claim 1, 3, or 5, wherein the nucleotide arrays are selected from the group consisting of arrays having spotted thereon cDNA sequences and arrays having synthesized thereon oligonucleotides.
